(19) United States
(12) Patent Application Publication
(10) Pub. No.: US 2022/0160649 A1
(43) Pub. Date: May 26, 2022

(54) PHARMACEUTICAL COMPOSITION

(71) Applicant: Rita DOBMEYER, Hergiswil (CH)

(72) Inventor: Rita DOBMEYER, Hergiswil (CH)

(21) Appl. No.: 17/441,153

(22) PCT Filed: Mar. 13, 2020
(86) PCT No.: PCT/EP2020/057005

§ 371 Date: Sep. 20, 2021

(30) Foreign Application Priority Data
Mar. 20, 2019 (EP) 19164177.8
Jan. 31, 2020 (EP) 20155027.4

(51) Int. Cl. A61K 31/05; A61K 9/00; A61K 47/46; A61K 31/352; A61K 45/06; A61P 17/00

(52) U.S. Cl. CPC A61K 31/05; A61K 9/0014; A61P 17/00; A61K 31/352; A61K 45/06; A61K 47/46

(57) ABSTRACT

The invention relates to a pharmaceutical composition comprising fulvic acid, a resveratrol derivative, optionally at least one boron-containing compound or a pharmaceutically acceptable salt thereof, and a pharmaceutically acceptable excipient, in particular for the treatment or prevention of inflammatory skin disorders.

### CROSS-REFERENCE TO RELATED APPLICATIONS

This application claims the benefit of priority to European patent applications EP19164177.8 filed on Mar. 20, 2019 and EP 20155027.4, filed on Jan. 31, 2020; the entire content of this application is hereby incorporated by reference in its entirety.

### BACKGROUND OF THE INVENTION

### 1. Technical Field

The invention relates to a pharmaceutical composition comprising a fulvic acid, a resveratrol derivative, optionally a boron-containing compound, and a pharmaceutically acceptable excipient.

### 2. Prior Art

Humic substances are ubiquitous in nature and arise from the decay of plant and animal residues in the environment. These substances can be divided into humic acid, fulvic acid and humaic acid based on the solubility in water as a function of pH. Fulvic acid is the fraction that is soluble in water under all pH conditions and is in general lower in molecular size and weight and lower in color intensity than humic acids.

Humic substances commonly account for 50% of the dissolved organic carbon concentrations in stream water, of which 90 to 95% are fulvic acids. Humic acids are 3 to 5 times more abundant in soils than fulvic acids, whereas fulvic acids are 9 to 10 times more abundant in water than humic acids.

U.S. Pat. Nos. 4,999,202 and 5,204,368 disclose compositions having anti-bacterial and bacteriostatic properties containing a fulvic acid, salt or derivative thereof as the active ingredient. These compositions are described as being useful as disinfectants.

U.S. Pat. No. 6,659,500 suggests the use of fulvic acids for the treatment of inflammation, acne, eczema, bacterial infection, viral infection, or any combination thereof.

J J Gandy, J R Snyman, C E J van Rensburg, Clinical, Cosmetic and Investigational Dermatology 2011:4, 145-148 describes a study evaluating the efficacy and safety of fulvic acid in the treatment of eczema in patients 2 years and older.

A first group of boron-containing compounds comprises inorganic borates including boric acid B(OH)₃; this group includes a large number of boron-containing oxyanions. The term “borates” may also refer to tetrahedral boron anions, or more loosely to chemical compounds, which contain borate anions of either description. Larger borates are composed of trigonal planar BO₃ or tetrahedral BO₄ structural units, joined together via shared oxygen atoms and may be cyclic or linear in structure.

As an antibacterial compound, boric acid can also be used as an acne treatment. It is also used as prevention of athlete's foot, by inserting powder in the socks or stockings, and in alcohol solution can be used to treat some kinds of otitis externa (ear infection) in both humans and animals.

A second group of boron-containing compounds comprises organic small molecules, in which the boron atom is covalently linked to at least on carbon atom of a substituted carbohydryl group. A group of boron-containing small molecules is described in the International Patent application WO 2006/089067.

Resveratrol derivatives are known natural products also called stilbenoids, i.e. hydroxylated stilbenes including the methyl ethers and glycosides thereof, which are known to have various beneficial pharmaceutical properties.

The International patent application WO 2017/102565 suggests pharmaceutical compositions comprising a fulvic acid, at least one boron-containing compound or a pharmaceutically acceptable salt thereof, and a pharmaceutically acceptable excipient.

Despite many attempts to provide a medicament for the treatment or prevention of inflammatory skin disorders, there is still an unmet need for a highly efficient and safe medicament for these disorders in humans, farm and companion animals. Accordingly, the problem underlying the present invention is to provide a medicament, which alleviates or prevents efficiently inflammatory skin disorders.

### SHORT SUMMARY OF THE INVENTION

Surprisingly, it has been found, that a concurrent or subsequent administration of a resveratrol derivative and optionally a boron-containing compound enhances the efficacy of fulvic acids against inflammatory skin disorders.

Accordingly, the present invention relates to a pharmaceutical composition comprising a fulvic acid, a resveratrol derivative, optionally at least one boron-containing compound or a pharmaceutically acceptable salt thereof, and a pharmaceutically acceptable excipient.

Furthermore, the invention relates to a pharmaceutical kit for the preparation of such a pharmaceutical composition essentially consisting of
(A) a first compartment containing a pharmaceutical composition comprising a fulvic acid and a pharmaceutically acceptable excipient;
(B) a second compartment containing a pharmaceutical composition comprising a resveratrol derivative and a pharmaceutically acceptable excipient;
(C) optionally a third compartment containing a pharmaceutical composition comprising a boron-containing compound or a pharmaceutically acceptable salt thereof and a pharmaceutically acceptable excipient;
(D) a leaflet describing the dosage and administration of each of the pharmaceutical compositions (A), (B) and optional (C).

### BRIEF DESCRIPTION OF THE DRAWINGS

FIG. 1A is a photographic representation of male infant suffering from symmetric eczema lesions before treatment.

FIG. 1B is a photographic representation of the same male infant after twice-daily application of a composition according to the present invention.

### DETAILED DESCRIPTION OF THE INVENTION

The term “fulvic acid” as used hereinbefore and herein below relates to the fraction of humic acids that is soluble in water under all pH conditions and is in general lower in molecular size and weight and lower in color intensity than the remaining humic acids. Preferred fulvic acids are those, which are prepared from carbohydrates. Preferred are the “carbohydrate derived fulvic acids (CHD-FA)”, which are obtainable by a process, which is, for example, disclosed by U.S. Pat. No. 8,114,910 and comprises the following steps:

(a) subjecting a carbohydrate selected from the group consisting of a monosaccharide, a disaccharide, and a polysaccharide to wet oxidation to produce a reaction product comprising acidic components including fulvic acids in solution,

(b) treating the reaction product to remove substantially all of the acidic components having a molecular weight exceeding 20,000 Daltons;

wherein the wet oxidation includes the steps of producing a solution or suspension of the carbohydrate in water and subjecting the solution or suspension to elevated temperature and pressure conditions to oxidize the carbohydrate; where elevated temperature is in the range 100 to 300 degrees centigrade and the pressure is such that boiling of the water is prevented.

Most preferred is the compound of formula (I)

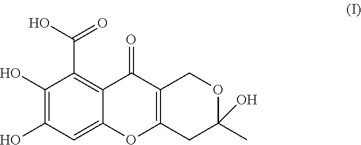 [SMILES: CC1(O)Cc2oc3cc(O)c(O)c(C(=O)O)c3c(=O)c2CO1]

The term “boron-containing compound” as used hereinbefore and herein below relates to inorganic boron containing compounds derived from boric acid such as boron nitride, borates, preferably boric acid as such, sodium borate or boron nitride (BN), in particular Caress® BN06, which is commercially available from Kobo Products Inc. South Plainfield, N.J. 07080. Furthermore preferred are organic boron-containing small molecule derived from boronic acid, wherein the boron atom is directly linked to at least one hydrocarbyl group. Many of these preferred boronic acid derivatives act as phosphodiesterase IV inhibitors, preferably those which comprise a 1,3-dihydro-1-hydroxy-2,1-benzoxaborol-5-yl group as a structural element.

Particularly preferred are such boron-containing compounds, which are compounds of formula (II),

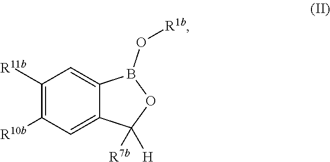

wherein

R^{7b} represents hydrogen, C₁₋₆ alkyl, preferably methyl or ethyl, or phenyl,

R^{10b} and R^{11b} each independently represent hydrogen, hydroxyl, amino, thiol or halogen, or an optionally substituted group selected from phenoxy, phenyl-C₁₋₆ alkoxy, phenylthio and phenyl-C₁₋₆ alkylthio, wherein the optional substituents are selected from the group consisting of hydroxyl, amino, thiol, halogen, cyano, nitro, C₁₋₆ alkyl, C₁₋₆ alkoxy, trifluoromethyl and difluoromethoxy;

R^{1b} represents hydrogen or a salt counter ion.

The most preferred boron-containing compound is 4-[(1,3-dihydro-1-hydroxy-2,1-benzoxaborol-5-yl)oxy]benzonitrile (pINN: crisaborole).

The term “resveratrol derivative” as used hereinbefore and herein below embraces the natural occurring hydroxylated stilbenes or stilbenoids including the methyl ethers and glycosides thereof. Preferred resveratrol derivatives are resveratrol, oxy-resveratrol also known as piceatannol, the methylated stilbenoids selected from the group consisting of 4-methoxyresveratrol, gnetucleistol D (2-methoxyoxyresveratrol), gnetucleistol E (3-methoxy-isorhapontigenin), isorhapontigenin (3,4′,5-trihydroxy-3′-methoxystilbene), pinostilbene (3-methoxyresveratrol), pterostilbene (3′,5′-dimethoxyresveratrol), rhapontigenin (piceatannol 4′-methyl ether), combretastatin A-1 and combretastatin A-4, and the glycosylated stilbenoids selected from the group consisting of piceid (trans-resveratrol-3-O-glucoside), trans-resveratrol-3-O-glucuronide, resveratroloside (trans-resveratrol-4′-O-beta-D-glucopyranoside), rhapontigenin 3-O-rutinoside, 4′-methoxy-(E)-resveratrol 3-O-rutinoside and rhaponticin (rhapontigenin glucoside).

Most preferred are resveratrol of formula (IIIA) and oxyresveratrol of formula (IIIB):

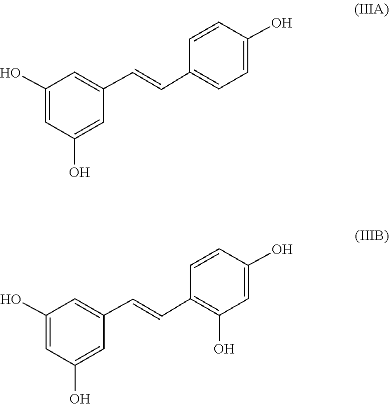 [SMILES: Oc1ccc(/C=C/c2cc(O)cc(O)c2)cc1 ; Oc1cc(O)cc(/C=C/c2ccc(O)cc2O)c1]

It is well known that resveratrol (IIIA) is almost insoluble in water and has stability issues. T. Petek et al., Molecules 2017, 22, 137; doi 10.3390 suggest to increase the solubility and stability of resveratrol in water with the aid of a dendrimer nanotechnology.

It has been found surprisingly that fulvic acid allows the preparation of stable water containing formulations of resveratrol for topical applications like gels, creams, ointments or the like.

The term “pharmaceutically acceptable salts” is meant to include salts of the compounds of the invention which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the present invention contain both basic and acidic functionalities that allow the compounds to be converted into either base or acid addition salts.

By “effective” amount of a drug, formulation, or permeant is meant a sufficient amount of a active agent to provide the desired local or systemic effect. A “Topically effective,” “Cosmetically effective,” “pharmaceutically effective,” or “therapeutically effective” amount refers to the amount of drug needed to effect the desired therapeutic result.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of each active ingredient that may be combined with the carrier materials to produce a single dosage form will vary depending upon the condition being treated and the particular mode of administration. Dosage unit forms will generally contain between from about 1 mg to about 500 mg of each of the active ingredients.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

A unit dose of the pharmaceutical composition according to the invention comprises preferably:

10 to 70% by weight, more preferably 30 to 60% by weight of fulvic acid, in particular the compound of formula (I);

optionally 0.01 to 0.10% by weight, more preferably 0.02 to 0.05% by weight of at least one boron containing compound or a pharmaceutically acceptable salt thereof, in particular boron nitride (BN); and

0.001 to 10% by weight, preferably 0.05 to 5% by weight, most preferably 0.2 to 2.5% by weight of a resveratrol derivative, preferably resveratrol or oxy-resveratrol, each with respect to the total weight of the composition.

The term “pharmaceutically acceptable excipient” or “pharmaceutically acceptable vehicle” refers to any formulation or excipient medium that provides the appropriate delivery of an effective amount of the active agents as defined herein, does not interfere with the effectiveness of the biological activity of the active agents, and that is sufficiently non-toxic to the host or patient. Representative excipients include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

“Pharmaceutically acceptable topical excipient” and equivalent terms refer to pharmaceutically acceptable excipient, as described herein above, suitable for topical application. An inactive liquid or cream vehicle capable of suspending or dissolving the active agent(s), and having the properties of being nontoxic and non-inflammatory when applied to the skin, nail, hair, claw or hoof is an example of a pharmaceutically acceptable topical carrier. This term is specifically intended to encompass excipient materials approved for use in topical cosmetics as well.

The term “topical administration” refers to the application of a pharmaceutical agent to the external surface of the skin, nail, hair, claw or hoof, such that the agent crosses the external surface of the skin, nail, hair, claw or hoof and enters the underlying tissues. Topical administration includes application of the composition to intact skin, nail, hair, claw or hoof, or to a broken, raw or open wound of skin, nail, hair, claw or hoof Topical administration of a pharmaceutical agent can result in a limited distribution of the agent to the skin and surrounding tissues or, when the agent is removed from the treatment area by the bloodstream, can result in systemic distribution of the agent.

The pharmaceutical formulations of the invention can take a variety of forms adapted to the chosen route of administration. Those skilled in the art will recognize various synthetic methodologies that may be employed to prepare non-toxic pharmaceutical formulations incorporating the fulvic acid and the boron containing compounds described herein. Those skilled in the art will recognize a wide variety of non-toxic pharmaceutically acceptable sol-vents that may be used to prepare solvates of the compounds of the invention, such as water, ethanol, propylene glycol, mineral oil, vegetable oil and dimethylsulfoxide (DMSO).

The compositions of the invention may be administered orally, topically, parenterally, by inhalation or spray or rectally in dosage unit formulations containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. The term parenteral as used herein includes sub-cutaneous injections.

In a preferred embodiment, the pharmaceutical compositions of the invention can be administered through the topical application of the fulvic acid, the resveratrol derivative and the boron containing compounds described herein.

The compositions of the present invention comprises fluid or semi-solid vehicles that may include but are not limited to polymers, thickeners, buffers, neutralizers, chelating agents, preservatives, surfactants or emulsifiers, antioxidants, waxes or oils, emollients, sunscreens, and a solvent or mixed solvent system. The solvent or mixed solvent system is important to the formation because it is primarily responsible for dissolving the fulvic acid, the resveratrol derivative and the boron-containing compound. The best solvent or mixed solvent systems are also capable of maintaining clinically relevant levels of the drug in solution despite the addition of a poor solvent to the formulation. The topical compositions useful in the subject invention can be made into a wide variety of product types. These include, but are not limited to, lotions, creams, gels, sticks, sprays, ointments, pastes, foams, mousses, and cleansers. These product types can comprise several types of carrier systems including, but not limited to particles, nanoparticles, and liposomes. If desired, disintegrating agents can be added, such as the cross-linked polyvinyl pyrrolidone, agar or alginic acid or a salt thereof such as sodium alginate. Techniques for formulation and administration can be found in Remington: The Science and Practice of Pharmacy, supra. The formulation can be selected to maximize delivery to a desired target site in the body.

Lotions, which are preparations that are to be applied to the skin, nail, hair, claw or hoof surface without friction are typically liquid or semi-liquid preparations in which finely divided solid, waxy, or liquid are dispersed. Lotions will typically contain suspending agents to produce better dispersions as well as compounds useful for localizing and holding the active agents in contact with the skin, nail, hair, claw or hoof, e.g., methylcellulose. sodium carboxymethyl-cellulose or the like.

Creams containing the active agent for delivery according to the present invention are viscous liquid or semisolid emulsions, either oil-in-water or water-in-oil. Cream bases are water-washable, and contain an oil phase, an emulsifier and an aqueous phase. The oil phase is generally comprised of petrolatum or a fatty alcohol, such as cetyl- or stearyl alcohol; the aqueous phase usually, although not necessarily, exceeds the oil phase in volume and generally contains a humectant. The emulsifier in a cream formulation, as explained in Remington: The Science and Practice of Pharmacy, supra, is generally a non-ionic, anionic, cationic or amphoteric surfactant.

Gel formulation can also be used in connection with the present invention. As will be appreciated by those working in the field of topical drug formulation gels are semisolid. Single-phase gels contain organic macromolecules distributed substantially uniformly throughout the carrier which is typically aqueous, but also may be a solvent or solvent blend.

Ointments, which are semisolid preparations, are typically based on petrolatum or other petroleum derivatives. As will be appreciated by the ordinarily skilled artisan, the specific ointment base to be used is one that provides for optimum delivery for the active agent chosen for a given formulation, and, preferably, provides for other desired characteristics as well, e.g., emolliency or the like. As with other carriers or vehicles, an ointment base should be inert, stable, non-irritating and non-sensitizing. As explained in Remington: The Science and Practice of Pharmacy. 19^{th} Ed. (Easton, Pa.: Mack Publishing Co., 1995), pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include for example, cetyl alcohol, glyceryl monostearate, lanolin and stearic acid. Preferred water-soluble ointment bases are prepared from polyethylene glycols of varying molecular weight; again, reference may be made to Remington: The Science and Practice of Pharmacy, supra, for further information.

Useful formulation of the invention also encompass sprays. Sprays generally provide the active agent in an aqueous and/or alcoholic solution which can be misted onto the skin, nail, hair, claw or hoof for delivery. Such sprays include those formulated to provide for concentration of the active agents solution at the site of administration following delivery, e.g., the spray solution can be primarily composed of alcohol or other like volatile liquid in which the drug or active agent can be dissolved. Upon delivery to the skin, nail, hair, claw or hoof, the carrier evaporates, leaving concentrated active agent at the site of administration.

The topical pharmaceutical compositions may also comprise suitable solid or gel phase carriers. Examples of such carriers include but are not limited to calcium carbonate, calcium phosphate, various sugars, starches, cellulose derivatives, gelatin, and polymers such as polyethylene glycols.

The topical pharmaceutical compositions may also comprise a suitable emulsifier, which refers to an agent that enhances or facilitates mixing and suspending oil-in-water or water-in-oil. The emulsifying agent used herein may consist of a single emulsifying agent or may be a nonionic, anionic, cationic or amphoteric surfactant or blend of two or more such surfactants; preferred for use herein are nonionic or anionic emulsifiers. Such surface-active agents are described in “McCutcheon's Detergent and Emulsifiers,” North American Edition, 1980 Annual published by the McCutcheon Division, MC Publishing Company, 175 Rock Road, Glen Rock, N.J. 07452, USA.

Preferred for use herein are high molecular weight alcohols such as cetearyl alcohol, cetyl alcohol, stearyl alcohol, emulsifying wax, glyceryl monostearate. Other examples are ethylene glycol distearate, sorbitan tristearate, propylene glycol monostearate, sorbitan monooleate, sorbitan monostearate (SPAN 60), diethylene glycol monolaurate, sorbitan monopalmitate, sucrose dioleate, sucrose stearate (CRODESTA F-160), polyoxyethylene lauryl ether (BRIJ 30), polyoxyethylene (2) stearyl ether (BRIJ 72), polyoxyethylene (21) stearyl ether (BRIJ 721), polyoxyethylene monostearate (Myrj 45), polyoxyethylene sorbitan monostearate (TWEEN 60), polyoxyethylene sorbitan monooleate (TWEEN 80), polyoxyethylene sorbitan monolaurate (TWEEN 20) and sodium oleate. Cholesterol and cholesterol derivatives may also be employed in externally used emulsions and promote w/o emulsions.

Especially suitable nonionic emulsifying agents are those with hydrophile-lipophile balances (HLB) of about 3 to 6 for w/o system and 8 to 18 for o/w system as determined by the method described by Paul L. Lindner in “Emulsions and Emulsion”, edited by Kenneth Lissant, published by Dekker, New York, N.Y., 1974, pages 188-190. More preferred for use herein are one or more nonionic surfactants that produce a system having HLB of about 8 to about 18.

Examples of such nonionic emulsifiers include but are not limited to “BRIJ 72”, the trade name for a polyoxyethylene (2) stearyl ether having an HLB of 4.9; “BRIJ 721”, the trade name for a polyoxyethylene (21) stearyl ether having an HLB of 15.5, “Brij 30”, the trade name for polyoxyethylene lauryl ether having an HLB of 9.7; “Polawax”, the trade name for emulsifying wax having an HLB of 8.0; “Span 60”, the trade name for sorbitan monostearate having an HLB of 4.7; “Crodesta F-160”, the trade name for sucrose stearate” having an HLB of 14.5. All of these materials are available from Ruger Chemicals Inc.; Croda; ICI Americas, Inc.; Spectrum Chemicals; and BASF. When the topical formulations of the present invention contain at least one emulsifying agent, each emulsifying agent is present in amount from about 0.5 to about 2.5 wt %, preferably 0.5 to 2.0%, more preferably 1.0% or 1.8%. Preferably the emulsifying agent comprises a mixture of steareth 21 (at about 1.8%) and steareth 2 (at about 1.0%).

The topical pharmaceutical compositions may also comprise suitable emollients. Emollients are materials used for the prevention or relief of dryness, as well as for the protection of the skin, nail, hair, claw or hoof Useful emollients include, but are not limited to, cetyl alcohol, isopropyl myristate, stearyl alcohol, and the like. A wide variety of suitable emollients are known and can be used herein. See e.g., Sagarin, Cosmetics, Science and Technology, 2nd Edition, Vol. 1, pp. 32-43 (1972), and U.S. Pat. No. 4,919,934, to Deckner et al., issued Apr. 24, 1990, both of which are incorporated herein by reference in their entirety. These materials are available from Ruger Chemical Co, (Irvington, N.J.).

When the topical formulations of the present invention contain at least one emollient, each emollient is present in an amount from about 0.1 to 15%, preferably 0.1 to about 3.0, more preferably 0.5, 1.0, or 2.5 wt %. Preferably, the emollient is a mixture of cetyl alcohol, isopropyl myristate and stearyl alcohol in a 1/5/2 ratio. The emollient may also be a mixture of cetyl alcohol and stearyl alcohol in a 1/2 ratio.

The topical pharmaceutical compositions may also comprise suitable antioxidants, substances known to inhibit oxidation. Antioxidants suitable for use in accordance with the present invention include, but are not limited to, butylated hydroxytoluene, ascorbic acid, sodium ascorbate, calcium ascorbate, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols such as vitamin E, and the like, including pharmaceutically acceptable salts and esters of these compounds. Preferably, the antioxidant is butylated hydroxytoluene, butylated hydroxyanisole, propyl gallate, ascorbic acid, pharmaceutically acceptable salts or esters thereof, or mixtures thereof. Most preferably, the antioxidant is butylated hydroxytoluene. These materials are available from Ruger Chemical Co, (Irvington, N.J.).

When the topical formulations of the present invention contain at least one antioxidant, the total amount of antioxidant present is from about 0.001 to 0.5 wt. %, preferably 0.05 to about 0.5 wt. %, more preferably 0.1 wt. %.

The topical pharmaceutical compositions may also comprise suitable preservatives. Preservatives are compounds added to a pharmaceutical formulation to act as an anti-microbial agent. Among preservatives known in the art as being effective and acceptable in parenteral formulations are benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. See, e.g., Wallhausser, K.-H., Develop. Biol. Standard, 24:9-28 (1974) (S. Krager, Basel). Preferably, the preservative is selected from methylparaben, propylparaben and mixtures thereof. These materials are available from Inolex Chemical Co (Philadelphia, Pa.) or Spectrum Chemicals.

When the topical formulations of the present invention contain at least one preservative, the total amount of preservative present is from about 0.01 to about 0.5 wt %, preferably from about 0.1 to 0.5 wt %, more preferably from about 0.03 to about 0.15 wt %. Preferably the preservative is a mixture of methylparaben and proplybarben in a 5/1 ratio. When alcohol is used as a preservative, the amount is usually 15 to 20 wt %.

The topical pharmaceutical compositions may also comprise suitable chelating agents to form complexes with metal cations that do not cross a lipid bilayer. Examples of suitable chelating agents include ethylene diamine tetraacetic acid (EDTA), ethylene glycol-bis(beta-aminoethyl ether)-N,N,N′,N′-tetraacetic acid (EGTA) and 8-Amino-2-[(2-amino-5-methylphenoxy)methyl]-6-methoxyquinoline-N,N,N′,N′-tetraacetic acid, tetrapotassium salt (QUIN-2). Preferably the chelating agents are EDTA and citric acid. These materials are available from Spectrum Chemicals.

When the topical formulations of the present invention contain at least one chelating agent, the total amount of chelating agent present is from about 0.005% to 2.0% by weight, preferably from about 0.05% to about 0.5 wt. %, more preferably about 0.10% by weight.

The topical pharmaceutical compositions may also comprise suitable neutralizing agents used to adjust the pH of the formulation to within a pharmaceutically acceptable range. Examples of neutralizing agents include but are not limited to trolamine, tromethamine, sodium hydroxide, hydrochloric acid, citric acid, and acetic acid.

When the topical formulations of the present invention contain at least one neutralizing agent, the total amount of neutralizing agent present is from about 0.1 wt. % to about 10 wt. %, preferably 0.1 wt. % to about 5.0 wt. %, and more preferably about 1.0 wt. %. The neutralizing agent is generally added in whatever amount is required to bring the formulation to the desired pH.

The topical pharmaceutical compositions may also comprise suitable viscosity increasing agents. These components are diffusible compounds capable of increasing the viscosity of a polymer-containing solution through the interaction of the agent with the polymer. CARBOPOL ULTREZ 10 may be used as a viscosity-increasing agent.

When the topical formulations of the present invention contain at least one viscosity increasing agent, the total amount of viscosity increasing agent present is from about 0.25% to about 5.0% by weight, preferably from about 0.25% to about 1.0%, and more preferably from about 0.4% to about 0.6% by weight.

The topical pharmaceutical compositions may also comprise suitable nail penetration enhancers. Examples of nail penetration enhancers include mercaptan compounds, sulfites and bisulfites, keratolytic agents and surfactants. Nail penetration enhancers suitable for use in the invention are described in detail in Malhotra et al., J. Pharm. Sci., 91:2, 312-323 (2002), which is incorporated herein by reference in its entirety.

The topical pharmaceutical compositions may also comprise suitable one or more fragrants in order to mask the smell of the fulvic acid. Examples of such fragrants include synthetic or natural essential oils such as lemon oil, orange oil, in particular bitter orange flower oil, peppermint oil, spearmint oil, cedar wood oil, eucalyptus oil, rose oil, rosehip oil, clove oil, lavender essential oil, balsam of Peru, patchouli oil and sandalwood oil or mixtures thereof.

The invention relates furthermore to a topical pharmaceutical composition comprising 20.0 to 70.0% by weight of fulvic acid, a resveratrol derivative, one or more fragrants selected from the group consisting of natural and synthetic essential oils, optionally one or more boron containing compound and a pharmaceutically acceptable excipient. As a rule 0.01 to 5.0 wt.-% of one or more fragrants, preferably 0.1 to 2.5 wt.-% of one or two essential oils will suffice to mask the unpleasant smell of the fulvic acid. Most preferred the topical composition of the present invention essentially consists of
15 to 50 wt.-%, in particular 20 to 40 wt.-% of carbohydrate derived fulvic acid of formula (I);
0.5 to 5.0 wt.-% in particular 0.7 to 1.5 wt.-% of resveratrol of formula (IIIA);
0.2 to 3.0 wt.-% in particular 0.3 to 2.5 wt.-% of one or two fragrants selected from the group of natural essential oils, in particular rosehip oil;
optionally 0.010% to 10% of another cosmetic or pharmaceutical active ingredient; and
45 to 85 wt.-%, in particular 55 to 75 wt.-% of semifluid base comprising one or more components selected from the group consisting of polar hydrophilic and non-polar hydrophobic solvents, thickeners, emulsifiers, emollient, carriers, preservatives, penetration enhancers, antioxidant and neutralizing agents.

The topical pharmaceutical compositions may also comprise one or more suitable solvents. The ability of any solid substance (solute) to dissolve in any liquid substance (solvent) is dependent upon the physical properties of the solute and the solvent. When solutes and solvents have similar physical properties, the solubility of the solute in the solvent will be the greatest. This gives rise to the traditional understanding that “like dissolves like.” Solvents can be characterized in one extreme as non-polar, lipophilic oils, while in the other extreme as polar hydrophilic solvents. Oily solvents dissolve other non-polar substances by Van der Wals interactions while water and other hydrophilic solvents dissolve polar substances by ionic, dipole, or hydrogen bonding interactions. All solvents can be listed along a continuum from the least polar, i.e. hydrocarbons such as decane, to the most polar solvent being water. A solute will have its greatest solubility in solvents having equivalent polarity. Thus, for drugs having minimal solubility in water, less polar solvents will provide improved solubility with the solvent having polarity nearly equivalent to the solute providing maximum solubility. Most drugs have intermediate polarity, and thus experience maximum solubility in solvents such as propylene glycol or ethanol, which are significantly less polar than water. If the drug has greater solubility in propylene glycol (for example 8% (w/w)) than in water (for example 0.1% (w/w)), then addition of water to propylene glycol should decrease the maximum amount of drug solubility for the solvent mixture compared with pure propylene glycol. Addition of a poor solvent to an excellent solvent will decrease the maximum solubility for the blend compared with the maximum solubility in the excellent solvent.

When compounds are incorporated into topical formulations, the concentration of active ingredient in the formulation may be limited by the solubility of the active ingredient in the chosen solvent and/or carrier. Non-lipophilic drugs typically display very low solubility in pharmaceutically acceptable solvents and/or carriers. For example, the solubility of some compounds in the invention in water is less than 0.00025% wt/wt. The solubility of the same compounds in the invention can be less than about 2% wt/wt in either propylene glycol or isopropyl myristate. In one embodiment of the present invention, diethylene glycol monoethyl ether (DGME) is the solvent used to dissolve the compounds of formula (I) and of formula (II). In another embodiment, a DGME water co-solvent system is used to dissolve the compounds of formula (I) and of formula (II). The solvent capacity of DGME drops when water is added; however, the DGME/water co-solvent system can be designed to maintain the desired concentration of from about 0.1% to about 5% wt./wt. of both the active ingredients. Preferably, the active ingredients are present from about 0.5% to about 3% wt./wt., and more preferably at about 1% wt./wt., in the as-applied topical formulations. Because DGME is less volatile than water, as the topical formulation evaporates upon application, the active agent becomes more soluble in the cream formulation. This increased solubility reduces the likelihood of reduced bioavailability caused by the drug precipitating on the surface of the skin, nail, hair, claw or hoof.

Liquid forms, such as lotions suitable for topical administration or suitable for cosmetic application, may include a suitable aqueous or non-aqueous vehicle with buffers, suspending and dispensing agents, thickeners, penetration enhancers, and the like. Solid forms such as creams or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsoms, micro-sponges and the like.

Additionally, the compounds can be delivered using a sustained-release system, such as semipermeable matrices of solid hydrophobic polymers containing the therapeutic agent. Various sustained-release materials have been established and are well known by those skilled in the art.

Topical treatment regimens according to the practice of this invention comprise applying the composition directly to the skin, nail, hair, claw or hoof at the application site, from one to several times daily.

Formulations of the present invention can be used to treat, ameliorate or prevent conditions or symptoms associated with inflammatory skin disorders such as acne, atopic dermatitis, eczema, rashes or psoriasis and the like.

In an exemplary embodiment, the pharmaceutical formulation includes a simple solution. In an exemplary embodiment, the simple solution includes an alcohol. In an exemplary embodiment, the simple solution includes alcohol and water. In an exemplary embodiment, the alcohol is ethanol, ethylene glycol, propanol, polypropylene glycol, isopropanol or butanol.

In another exemplary embodiment, the simple solution is a member selected from about 10% polypropylene glycol and about 90% ethanol; about 20% polypropylene glycol and about 80% ethanol; about 30% polypropylene glycol and about 70% ethanol; about 40% polypropylene glycol and about 60% ethanol; about 50% polypropylene glycol and about 50% ethanol; about 60% polypropylene glycol and about 40% ethanol; about 70% polypropylene glycol and about 30% ethanol; about 80% polypropylene glycol and about 20% ethanol; about 90% polypropylene glycol and about 10% ethanol.

In an exemplary embodiment, the pharmaceutical formulation is a lacquer. Please see Remington's, supra, for more information on the production of lacquers.

In an exemplary embodiment, fulvic acid and the optional boron-containing compound are each present in said pharmaceutical formulation in a concentration of from about 0.01% to about 70%. In an exemplary embodiment, fulvic acid and the boron-containing compound are each present in said pharmaceutical formulation in a concentration of from about 0.02% to about 60%. In an exemplary embodiment, fulvic acid and the boron-containing compound are each present in said pharmaceutical formulation in a concentration of from about 0.05% to about 55%. In an exemplary embodiment, fulvic acid and the boron-containing compound are each present in said pharmaceutical formulation in a concentration of from about 0.1% to about 52%. In an exemplary embodiment, fulvic acid and the boron-containing compound are each present in said pharmaceutical formulation in a concentration of from about 0.2% to about 51%. In an exemplary embodiment, fulvic acid and the boron-containing compound are each present in said pharmaceutical formulation in a concentration of from about 0.3% to about 50%.

In an exemplary embodiment, 0.001 to 10% by weight of a resveratrol derivative are present in a pharmaceutical unit dose according to the invention. In an exemplary embodiment, 0.05 to 5% by weight of a resveratrol derivative are present in a pharmaceutical unit dose according to the invention. In an exemplary embodiment, 0.1 to 4% by weight of a resveratrol derivative are present in a pharmaceutical unit dose according to the invention. In an exemplary embodiment, 0.15 to 3.5% by weight of a resveratrol derivative are present in a pharmaceutical unit dose according to the invention. In an exemplary embodiment, 0.2 to 2.5% by weight of a resveratrol derivative are present in a pharmaceutical unit dose according to the invention.

In another aspect, the invention provides a pharmaceutical composition or method for the treatment or prevention of an inflammatory skin disorder or disease. The method includes administering to the patient a therapeutically effective amount of the pharmaceutical composition of the invention, sufficient to treat or prevent said disorder. In an exemplary embodiment, the composition of the invention comprises the compound of formula (I), optionally a boron compound selected from the group consisting of boron nitride, boric acid, sodium borate or a compound of formula (II), in particular crisaborole and a resveratrol derivative, in particular resveratrol of formula (IIIA) or oxy-resveratrol of formula (IIIB). In another exemplary embodiment, the patient is a member selected from human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the patient is a human. In another exemplary embodiment, the patient is a member selected from a human, cattle, goat, pig, sheep, horse, cow, bull, dog, guinea pig, gerbil, rabbit, cat, chicken and turkey. Most preferably the patient is a human of any gender or age. Since all the components of the pharmaceutical composition of the invention are compatible for children and young adults, it can be used for the treatment of paediatric patients. The term “paediatric patients” as used hereinbefore and hereinbelow includes new-born babies, toddlers, infants, children and young adults, which suffer from skin disorders. Preferably the pharmaceutical composition of the present invention can be used to treat or prevent skin disorders selected from the group consisting of perioral dermatitis, seborrheic dermatitis and candidiasis, in particular diaper rash.

In another exemplary embodiment, the disorder is a member selected from a systemic infection, a cutaneous infection, and an ungual or periungual infection.

The following are examples of the cosmetic and pharmaceutical agents, such as an anti-inflammatory agent, antifungal agent, vitamin, anti-aging agent, sunscreen, and/or acne-treating agent that can be added to the topical pharmaceutical formulations of the present invention. The following agents are known compounds and are readily available commercially.

Anti-inflammatory agents include, but are not limited to, bisabolol, mentholatum, dapsone, aloe, hydrocortisone, and the like.

Antifungal agents include, but are not limited to, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, nitroxoline and the like.

Vitamins include, but are not limited to, Vitamin B, Vitamin E, Vitamin A, Vitamin D, and the like and vitamin derivatives such as tazarotene, calcipotriene, tretinoin, adapalene and the like.

Anti-aging agents include, but are not limited to, niacinamide, retinol and retinoid derivatives, AHA, Ascorbic acid, lipoic acid, coenzyme Q 10, beta hydroxy acids, salicylic acid, copper binding peptides, dimethylaminoethyl (DAEA), and the like.

UV filters, sunscreens and/or sunburn relief agents include, but are not limited to, ylmethoxydibenzoyl ethane, 2-(4-ethoxy-anilinomethylene)-propanedioic acid diethyl ester, ethylhexylmethoxy-cinnamate, ethylhexyl salicylate, octocrylene, 2-phenylbenzimidazole-5-sulphonic acid, dimethico diethylbenzalmalonate, 2,4-bis((4-(ethyl-hexyloxy)-2-hydroxy)-phenyl)-6-(4-methoxyphenyl)-1,3,5-triazine, titan dioxide, zinc dioxide, PABA, jojoba, aloe, padimate-O, methoxycinnamates, proxamine HCl, lidocaine and the like. Sunless tanning agents include, but are not limited to, dihydroxyacetone (DHA).

Psoriasis-treating agents and/or acne-treating agents include, but are not limited to, salicylic acid, benzoyl peroxide, coal tar, selenium sulfide, zinc oxide, pyrithione (zinc and/or sodium), tazarotene, calcipotriene, tretinoin, adapalene and the like.

Agents that are effective to control or modify keratinization, including without limitation: tretinoin, tazarotene, and adapalene.

The compositions comprising the active agents of formula (I) and of formula (II), and optionally at least one of these additional agents, are preferably to be administered topically. In a primary application, this leads to the pharmaceutical composition of the invention and any other active agent working upon and treating the skin, nail, hair, claw or hoof. Alternatively, any one of the topically applied active agents may also be delivered systemically by transdermal routes.

In such compositions an additional cosmetically or pharmaceutically effective agent, such as an anti-inflammatory agent, vitamin, anti-aging agent, sunscreen, and/or acne-treating agent, for example, is usually a minor component (from about 0.001% to about 20% by weight or preferably from about 0.01% to about 10% by weight) with the remainder being various vehicles or carriers and processing aids helpful for forming the desired dosing form.

### EXEMPLIFICATION

The invention now being generally described, will be more readily understood by reference to the following Examples, which are included merely for purposes of illustration of certain aspects and embodiments of the present invention, and are not intended to limit the invention. All publications and patents mentioned herein are hereby incorporated by reference in their entirety as if each individual publication or patent was specifically and individually indicated to be incorporated by reference. In case of conflict, the present application, including any definitions herein, will control.

### Example 1

Topical Formulations

Fulvic acid, the resveratrol derivative and the boron-containing compounds of the present invention can be administered to a patient using a therapeutically effective amount of the carbohydrate derived fulvic acid of the formula (I), optionally a boron compound such as boron nitride or the compound of formula (II) and resveratrol of formula (IIIA) in any one of the following formulations. The following formulations (A), (B) and (C) combine good durability with good ease of use.

(A) Thin Cream (Lotion)

| Phase | INCI Name | Quantity (g) |
|---|---|---|
| A | Water (Aqua) | 145.95 |
| A | Glycerin | 40 |
| A | Sophora Flavescens Extract | 0.03 |
| A | Maltodextrin & Aloe Ferox Leaf Extract | 10 |
| A1 | Xanthan Gum | 5 |
| B | Cannabis Sativa (Hemp) Seed Oil | 50 |
| B | Butyrospermum Parkii (Shea) Butter | 50 |
| B | Isoamyl Laurate | 20 |
| B | Linum usitatissimum (Linseed) Seed Oil | 30 |
| B | Cetyl Alcohol | 30 |
| B | Glyceryl Stearate Citrate | 30 |
| B | Boron Nitride | 3 |
| B | Resveratrol of formula (IIIA) | 50 |
| B | Tocopherol & Helianthus Annuus (Sunflower) Seed Oil | 1 |
| C | Bulbine Frutescens Extract & Alcohol | 0.02 |
| D | Fulvic Acid of formula (I) | 500 |
| E | Sodium Citrate | 30 |
| F | Pogostemon Cablin (Patchouli) Leaf Oil | 1 |
| F | Citrus aurantium amara (Bitter Orange) Flower Oil | 4 |
| Total | | 1000.00 |

Method:
a) Phase A and B are added to clean mixing vessels and heat to 70-75° C.
b) Phase A1 is added to phase A, and homogenised until xanthan gum is completely dispersed (approx. 5-10 minutes).
c) Phase B is stirred into phase A and kept stirring for 5 minutes to emulsify.
d) The mixture is homogenised for approx. 3 minutes at 3000 rpm
e) The mixture is cooled down under medium stirring.
g) Phase D is added, below 40° C., to main vessel and mixed until homogenous.
h) pH is adjust to recommended pH value with sodium citrate.
i) Phase F is added and mixed until homogenous.

| Specific Values: | |
|---|---|
| Appearance: | Thin lotion |
| Colour: | Off-white |
| pH: | 4.0-4.5 |
| Odour: | Neroli/woody |

(B) Thick Cream

| Phase | INCI Name | Quantity (g) |
|---|---|---|
| A | Water (Aqua) | 126.95 |
| A | Glycerin | 50 |
| A | Sophora Flavescens Extract | 0.03 |
| A | Maltodextrin & Aloe Ferox Leaf Extract | 10 |
| A1 | Xanthan Gum | 5 |
| B | Cannabis Sativa (Hemp) Seed Oil | 55 |
| B | Butyrospermum Parkii (Shea) Butter | 65 |
| B | Isoamyl Laurate | 20 |
| B | Linum usitatissimum (Linseed) Seed Oil | 30 |
| B | Cetyl Alcohol | 20 |
| B | Glyceryl Stearate Citrate | 30 |
| B | Boron Nitride | 3 |
| B | Resveratrol of formula (IIIA) | 50 |
| B | Tocopherol & Helianthus Annuus (Sunflower) Seed Oil | 1 |
| C | Bulbine Frutescens Extract & Alcohol | 0.02 |
| D | Fulvic Acid of formula (I) | 500 |
| E | Sodium Citrate | 30 |
| F | Pogostemon Cablin (Patchouli) Leaf Oil | 1 |
| F | Santalum Album (Sandalwood) | 3 |
| Total | | 1000.00 |

Method:

a) Phases A & B are added to clean mixing vessels and heat to 70-75° C.

b) Phase A1 is added to phase A, and homogenised until xanthan gum is completely dispersed (approx. 5-10 minutes).

c) Phase B is added into A under medium stirring and kept stirring for 5 minutes to emulsify.

d) The mixture is homogenised for approx. 3 min. at 3000 rpm.

e) It is cooled down under medium stirring.

g) Phase D is added, below 40° C., to main vessel and mixed until homogenous.

h) pH is adjusted to recommended pH value with sodium citrate.

i) Add phase F and mix until homogenous.

| Spec Values: | |
|---|---|
| Appearance: | Thick Cream |
| Colour: | Light cream |
| pH: | 4.0-4.5 |
| Odour: | Woody |

(C) Ointment

| INCI Name | Quantity (wt.-%) |
|---|---|
| Fulvic Acid of formula (I) | 30.0 |
| Resveratrol of formula (IIIA) | 1.0 |
| Calcium D- pantothenate | 1.0 |
| Ascorbic acid | 0.2 |
| Alpha - tocopherol | 0.2 |
| Rosehip oil | 2.0 |
| Sodium acetate | 1.0 |
| Potassium citrate | 0.6 |
| Unguentum emulsificans aquosum | 50.0 |
| Glycerine | 15.0 |

The method to prepare these formulations is well known in the art and is found in references such as Remington: The Science and Practice of Pharmacy, supra.

### Example 2

In a patient having 2 different sites of inflammatory episodes at the skin of the chest treatment is started in one half of the inflammatory side (I) with the 1 g of the thin cream of example 1 A containing 500 mg of fulvic acid, 3 mg of boron and 50 mg of resveratrol, the other side (II) is treated with 1 g of the thin cream of example 1 A of WO 2017/102565 containing 500 mg of fulvic acid and 3 mg of boron nitride, only.

After four identical doses of both the treatments (I) and (II) every other week, the patient shows remarkable improvement and his eczema area and severity index (EASI; Hanifin J. M et al. (2001) The eczema area and severity index (EASI): assessment of reliability in atopic dermatitis. EASI Evaluator Group. Exp Dermatol. February; 10(1):11-8.) improves 86% and the severity scoring of atopic dermatitis index (SCORAD; Kunz B, et al (1997). Clinical validation and guidelines for the SCORAD index: consensus report of the European Task Force on Atopic Dermatitis. Dermatology; 195(1):10-9.) improves 85% with treatment (I). The treatment (II) achieves an improvement of the EASI of only 43% and of the SCORAD of 44%.

The key quality of life issues are that his itching is nearly resolved and he is sleeping better at night and his eyebrows are re-growing.

The interim results are presented summarized in the following tables I and II.

### Example 3

In another patient having 2 different sites of inflammatory episodes at the skin of the chest, chest treatment is started in one half of the inflammatory side (III) with the 1 g of the ointment of example 1 (C) containing 300 mg of fulvic acid, and 1 mg of resveratrol, the other side (IV) is treated with 1 g of the thin cream of example 1 A of WO 2017/102565 containing 500 mg of fulvic acid and 3 mg of boron nitride, only.

After four identical doses of both the treatments (III) and (IV) every other week, the patient shows remarkable improvement and his eczema area and severity index improves 75% and the severity scoring of atopic dermatitis index improves 79% with treatment (III). The treatment (IV) achieves an improvement of the EASI of only 45% and of the SCORAD of 46%.

In addition, resveratrol has a mild anti-microbial activity without being an antibiotic. Therefore, eventually keeping the infected site cleaner.

A significant improvement in the local response to treatment is observed in the combination of fulvic acid/resveratrol and optionally boron nitride, when compared with the skin treated with fulvic acid and boron nitride, only.

### Example 4

Case Report to investigate the efficacy and tolerability of a 2 days treatment with twice daily topical administration of CHD-Fulvic Acid and Resveratrol ointment (cp. Example 1 (C)) during the neonatal period for facial eczema.

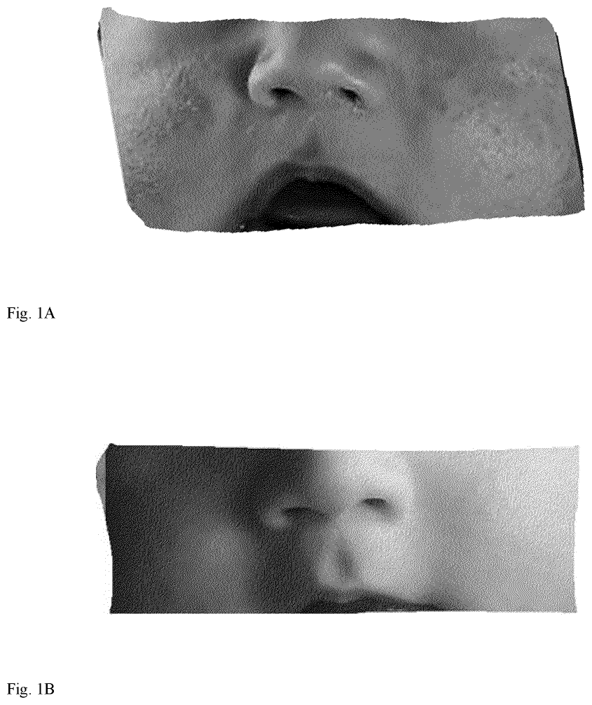

A 6-month-old male infant, with no known food allergy or intolerance, showed since more than 2 weeks with symmetric eczema lesions characterised by itchy, erythematous skin eruptions, no signs of bacterial superinfection on both cheeks (chin, forehead and scalp were not affected) supporting the diagnose of infantile eczema (cp. FIG. 1A). Prior treatment with emollients was not effective and produced discomfort.

After twice-daily application of the ointment of Example 1 (C) with the consent of his parents, in addition to withdrawal of emollients, for 2 days produced complete clinical cure already on day 2 with resolution of all clinical signs and symptoms as can be seen from FIG. 1B. In addition, improvements in all facial eczema lesions, scratching behaviour and sleep quality were reported. Eczema has been cured.

TABLE I
Modified SCORAD index

| | | | | | Week 6 | Week 6 |
|---|---|---|---|---|---|---|
| | | | Week 6 Score | Week 6 Score | % Improvement | % Improvement |
| | | | Treatment | | | |
| | | | fulvic acid + boron | fulvic acid + | fulvic acid + boron | fulvic acid + |
| | | Baseline | nitride + resveratrol | boron nitride | nitride + resveratrol | boron nitride |
| Affected Skin | | 4 | 1 | 2 | 75% | 50% |
| Area | | | | | | |
| Anterior trunk | | | | | | |
| (up to 18%) | | | | | | |
| Intensity (0-3) | Redness | 3 | 1 | 2 | 67% | 33% |
| | Swelling | 2 | 0 | 2 | 100% | 0% |
| | Oozing | 2 | 0 | 1 | 100% | 50% |
| | Scratch marks | 1 | 0 | 0 | 100% | 100% |
| | Skin thickening | 2 | 0 | 1 | 100% | 50% |
| | Dryness | 3 | 1 | 1 | 67% | 67% |
| Subjective | Prurit | 6 | 1 | 2 | 83% | 67% |
| symptoms | | | | | | |
| (0 to 10) | | | | | | |
| | Insomnia | 2 | 0 | 0 | 100% | 100% |
| TOTAL | | 54.3 | 8.2 | 30.4 | 85% | 44% |
| Modified | | | | | | |
| SCORAD index | | | | | | |
| 0-103 | | | | | | |

TABLE II
Modified EASI score

| | | | | | Week 6 | Week 6 |
|---|---|---|---|---|---|---|
| | | | Week 6 Score | Week 6 Score | % Improvement | % Improvement |
| | | | Treatment | | | |
| | | | fulvic acid +boron | fulvic acid + | fulvic acid + boron | fulvic acid + |
| | | Baseline | nitride + resveratrol | boron nitride | nitride +resveratrol | boron nitride |
| Trunk Area Score* | | 1 | 1 | 1 | 0% | 0% |
| Severity score | Redness (erythema, | 3 | 1 | 2 | 67% | 33% |
| (0-3) | inflammation) | | | | | |
| | Thickness (induration, | 2 | 0 | 1 | 100% | 50% |
| | papulation, swelling-acute | | | | | |
| | eczema) | | | | | |
| | Scratching (excoriation) | 1 | 0 | 0 | 100% | 100% |
| | Lichenification (lined skin, | 1 | 0 | 1 | 100% | 0% |
| | furrowing, prurigo nodules- | | | | | |
| | chronic eczema | | | | | |
| TOTAL Severity | | 7 | 1 | 4 | 86% | 43% |
| SCORE | | | | | | |
| TOTAL EASI Score | | 2.1 | 0.3 | 1.2 | 86% | 43% |
| 0-72 | | | | | | |

*Trunk Area Score
0 No active eczema in this region
1 1-9%
2 10-29%
3 30-49%
4 50-69%
5 70-89%
6 90-100%: the entire region is affected by eczema

1-15. (canceled)

16. A topical pharmaceutical composition comprising:
20% to 70% of at least one carbohydrate-derived fulvic acid,
resveratrol and/or oxy-resveratrol,
at least one fragrant chosen from natural and synthetic essential oils, and
at least one pharmaceutically acceptable excipient,
wherein all amounts are by weight, relative to the total weight of the topical pharmaceutical composition.

17. The topical pharmaceutical composition according to claim 16, comprising fulvic acid of formula (I):

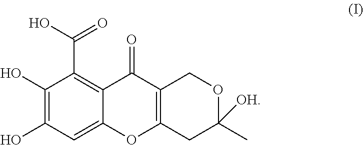 [SMILES: CC1(O)Cc2oc3cc(O)c(O)c(C(=O)O)c3c(=O)c2CO1]

18. The topical pharmaceutical composition according to claim 16, comprising resveratrol of formula (IIIA) and/or oxy-resveratrol of formula (IIIB):

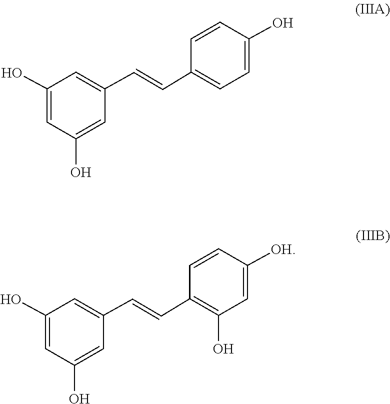 [SMILES: Oc1ccc(/C=C/c2cc(O)cc(O)c2)cc1 ; Oc1cc(O)cc(/C=C/c2ccc(O)cc2O)c1]

19. The topical pharmaceutical composition according to claim 16, comprising 0.001% to 10% of resveratrol and/or oxy-resveratrol.

20. The topical pharmaceutical composition according to claim 19, comprising 0.05% to 5% of resveratrol and/or oxy-resveratrol.

21. The topical pharmaceutical composition according to claim 20, comprising 0.2% to 2.5% of resveratrol and/or oxy-resveratrol.

22. The topical pharmaceutical composition according to claim 16, wherein the pharmaceutically acceptable excipient comprises at least one component chosen from polymers, thickeners, buffers, neutralizers, chelating agents, preservatives, surfactants, emulsifiers, antioxidants, waxes, oils, emollients, solvents, or penetration enhancers.

23. The topical pharmaceutical composition according to claim 16, wherein the pharmaceutically acceptable excipient comprises water, white petrolatum, propylene glycol, mono-glycerides, di-glycerides, paraffin, butylated hydroxytoluene, and/or edetate calcium disodium.

24. The topical pharmaceutical composition according to claim 16, consisting essentially of:
20% to 50% of carbohydrate-derived fulvic acid of formula (I): 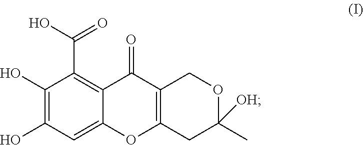 [SMILES: CC1(O)Cc2oc3cc(O)c(O)c(C(=O)O)c3c(=O)c2CO1]
0.5% to 5.0% of resveratrol of formula (IIIA): 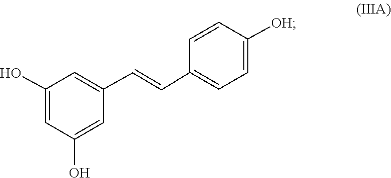 [SMILES: Oc1ccc(/C=C/c2cc(O)cc(O)c2)cc1]
0.2% to 3.0% of fragrants chosen from natural essential oils; and
45% to 85% of pharmaceutically acceptable excipients.

25. The topical pharmaceutical composition according to claim 24, consisting essentially of:
20% to 40% of carbohydrate derived fulvic acid of formula (I);
0.7% to 1.5% of resveratrol of formula (IIIA);
0.3% to 2.5% of fragrants chosen from natural essential oils; and
55% to 75% of pharmaceutically acceptable excipients.

26. The topical pharmaceutical composition according to claim 24, comprising rosehip oil.

27. The topical pharmaceutical composition according to claim 25, comprising rosehip oil.

28. The topical pharmaceutical composition according to claim 16, further comprising at least one boron-containing compound.

29. A method for treatment or prevention of a skin disorder in a patient in need thereof, comprising administering to the patient a topical pharmaceutical composition comprising:
20% to 70% of at least one carbohydrate-derived fulvic acid,
resveratrol and/or oxy-resveratrol,
at least one fragrant chosen from natural and synthetic essential oils, and
at least one pharmaceutically acceptable excipient,
wherein all amounts are by weight, relative to the total weight of the topical pharmaceutical composition.

30. The method according to claim 29, wherein the skin disorder is acne, atopic dermatitis, eczema, rashes, or psoriasis.

31. The method according to claim 29, wherein the patient in need thereof is a pediatric patient.

32. The method according to claim 31, wherein the skin disorder of the pediatric patient is perioral dermatitis, seborrheic dermatitis, or candidiasis.

33. The method according to claim 31, wherein the skin disorder of the pediatric patient is diaper rash.

34. A kit consisting essentially of:
(A) a first compartment containing a composition comprising 20% to 70% by weight of a carbohydrate derived fulvic acid (CHD-FA), at least one fragrant chosen from natural and synthetic essential oils, and at least one pharmaceutically acceptable excipient;
(B) a second compartment containing a composition comprising resveratrol and/or oxy-resveratrol derivative and a pharmaceutically acceptable excipient; and
(C) optionally, a leaflet describing the dosage and/or administration of the topical pharmaceutical compositions (A) and (B).
